SEQUENCE LISTING (12 sequences)

SEQ ID NO: 1 - protein, 12 aa, Artificial Sequence
  Description of Artificial Sequence  Synthetic Peptide
TETQEKNPLPSK

SEQ ID NO: 2 - DNA, 25 bp, Artificial Sequence
  Description of Artificial Sequence  Synthetic Primer
ccgatatggctgagatcgagaaatt

SEQ ID NO: 3 - DNA, 25 bp, Artificial Sequence
  Description of Artificial Sequence  Synthetic Primer
gagaagcaagctggcgaatcgtaat

SEQ ID NO: 4 - DNA, 25 bp, Artificial Sequence
  Description of Artificial Sequence  Synthetic Primer
tcaaagaatcagaactactgagcag

SEQ ID NO: 5 - DNA, 25 bp, Artificial Sequence
  Description of Artificial Sequence  Synthetic Primer
gggagatgatgaaatagagaggaaa

SEQ ID NO: 6 - DNA, 25 bp, Artificial Sequence
  Description of Artificial Sequence  Synthetic Primer
ccgggtaagtcctagagagataatt

SEQ ID NO: 7 - DNA, 25 bp, Artificial Sequence
  Description of Artificial Sequence  Synthetic Primer
gatccatgcagcgtatccgatgaat

SEQ ID NO: 8 - DNA, 25 bp, Artificial Sequence
  Description of Artificial Sequence  Synthetic Primer
tcataagagacatcaagtcgaacag

SEQ ID NO: 9 - DNA, 25 bp, Artificial Sequence
  Description of Artificial Sequence  Synthetic Primer
gggatgatgaaatagagaggagaaa

SEQ ID NO: 10 - protein, 13 aa, Artificial Sequence
  Description of Artificial Sequence  Synthetic Peptide
KTETQEKNPLPSK

SEQ ID NO: 11 - protein, 19 aa, Artificial Sequence
  Description of Artificial Sequence  Synthetic Peptide
TETQEKNPLPSKETIEQEK

SEQ ID NO: 12 - protein, 20 aa, Artificial Sequence
  Description of Artificial Sequence  Synthetic Peptide
KTETQEKNPLPSKETIEQEK

(19) United States
(12) Patent Application Publication
(10) Pub. No.: US 2006/0223122 A1
(43) Pub. Date: Oct. 5, 2006

(54) Classifying and predicting glomerulosclerosis using a proteomics approach

(76) Inventors: Agnes Fogo, Mt. Juliet, TN (US); Richard Caprioli, Brentwood, TN (US)

Correspondence Address: FULBRIGHT & JAWORSKI L.L.P., 600 CONGRESS AVE., SUITE 2400, AUSTIN, TX, 78701, US

(21) Appl. No.: 11/371,355

(22) Filed: Mar. 8, 2006

Related U.S. Application Data
(60) Provisional application No. 60/659,768, filed on Mar. 8, 2005.

(51) Int. Cl. G01N 33/567; G01N 33/543; G01N 33/53

(52) U.S. Cl. 435/7.2; 436/518; 436/86

(57) ABSTRACT

The present invention provides for a proteomic approach to classifying glomerular tissue as normal, non-sclerotic or sclerotic. In particular, the proteomic approach may employ laser capture microdissection followed by MALDI-TOF. A particular target of interest that is highly relevant in distinguishing such tissues is thymosin β4.

This application claims benefit of priority to Provisional Application U.S. Ser. No. 60/659,768, filed Mar. 8, 2005, the entire contents of which are hereby incorporated by reference in their entirety.

### BACKGROUND OF THE INVENTION

1. Field of the Invention

The present invention relates generally to the fields of protein biology and nephrology. More particularly, it concerns the classification of kidney tissue as normal, non-sclerotic or sclerotic based on the expression of various proteins identified as relevant to sclerosis.

2. Description of Related Art

Progression of focal segmental glomerulosclerosis (FSGS) is postulated to develop from early diffuse podocyte injury preceding overt sclerosis (Fogo et al., 1990; 2000), likely involving multiple complex mechanisms. With the advancement of proteomic techniques (Klein, 2004; Arthur, 2003), simultaneous examination of hundreds of proteins related to kidney disease holds promise in unraveling novel underlying mechanisms of progression and thus identifying possible targets for intervention in FSGS. The focal segmental nature of sclerosis raises the question of whether the remaining non-sclerotic glomeruli at a given time point in FSGS already are programmed to sclerotic pathways, or alternatively, have less prosclerotic activation and thus may be more susceptible to therapy.

To obtain protein expression profiles from a pure cell population, laser capture microdissection (LCM) has been combined with matrix-assisted laser desorption/ionization mass spectrometry (MALDI MS) for tissue protein profiling (Xu et al., 2002; Palmer-Toy et al., 2000). LCM is an important tool in biological research enabling the isolation of specific cell populations from a heterogeneous tissue section (Emmert-Buck et al., 1996). The technique of direct protein profiling from the laser capture microdissected cells using MALDI MS is fast, sensitive and accurate. This technique has been applied to several studies include human breast carcinoma (Xu et al., 2002), mouse epididymis (Chaurand et al., 2003) and human lung carcinoma (Bhattacharya et al., 2003). This technique enables one to obtain protein expression profiles from even minute structure within limited tissue samples. However, the applicability of these techniques to any given tissue or disease state must be determined empirically.

### SUMMARY OF THE INVENTION

Thus, in accordance with the present invention, there is provided a method of classifying glomerular tissue as normal, non-sclerotic or sclerotic comprising (a) obtaining a glomerular tissue sample; (b) analyzing protein content of the tissue sample; (c) comparing the protein content of the tissue sample with a predetermined standard; and (d) classifying the glomerular tissue as normal, non-sclerotic or sclerotic. The glomerular tissue may be classified as focal segmental glomerulosclerotic. The sample may be from a mammal, such as a human. The method may further compris making a medical treatment decision based on the classification.

Analyzing protein content may comprise assessing proteomic patterns, such as by mass spectrometry, immunohistochemistry or 2-D gel electrophoresis. Analyzing protein content may more particularly comprise assessing thymosin β4 expression, again, by using mass spectrometry or immunohistochemistry. Analyzing protein content may also comprise laser capture microdissection coupled with matrix-assisted laser desporption/ionization time-of-flight mass spectrometry. Analyzing may comprise assessing expression of one or more proteins having molecular weights of 4222 Daltons, 5485 Daltons, 7018 Daltons and 12,131 Daltons, which may be performed in conjunction with analyzing thymosin β4 expression.

In another embodiment, there is provided a method of identifying a marker in a glomerular tissue comprising (a) obtaining a diseased glomerular tissue sample; (b) analyzing protein content of the tissue sample; (c) comparing the protein content of the tissue sample with a normal glomerular tissue sample; and (d) identifying a marker in the diseased glomerular tissue sample that is as normal, non-sclerotic or sclerotic. Analyzing may comprise mass spectrometry, immunohistochemistry or 2-D gel electrophoresis.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed in this specification can be implemented with respect to any method or composition of the invention, and vice versa. Furthermore, compositions and kits of the invention can be used to achieve methods of the invention.

Throughout this application, the term “about” is used to indicate that a value includes the inherent variation of error for the device, the method being employed to determine the value, or the variation that exists among the study subjects.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-C—The process of laser capture microdissection of a normal glomerulus. (FIG. 1A) The normal glomerulus can be clearly recognized. (FIG. 1B) The remaining tissue section after the glomerulus was microdissected. (FIG. 1C) The microdissected glomerulus on the LCM film.

FIGS. 2A-C—Mass spectra obtained from three groups of glomeruli. (FIG. 2A) normal baseline glomeruli obtained at the time of nephrectomy, (FIG. 2B) nonsclerotic glomeruli from 12 wks after ⅚ nephrectomy, and (FIG. 2C) sclerotic glomeruli from 12 wks after ⅚ nephrectomy.

FIGS. 3A-C—Hierarchical cluster analysis of three different groups of glomeruli based on the top differentially expressed protein patterns. (FIG. 3A) Total of 30 non-sclerotic glomeruli and 60 normal glomeruli protein profiles were accurately classified. (FIG. 3B) One out of 60 normal glomeruli and 60 sclerotic glomeruli protein profiles was misclassified. (FIG. 3C) Seven out of 30 non-sclerotic glomeruli and 60 sclerotic glomeruli protein profiles were misclassified.

FIG. 4—Proteomic pattern. The overall proteomic pattern demonstrates that non-sclerotic glomeruli are more similar to sclerotic glomeruli than to the normal glomeruli.

FIG. 5A-B—Identification of thymosin β4 as a marker for sclerosis. (FIG. 5A) The MALDI mass spectrum of a HPLC fraction that contains the target peak at 4963.76 m/z. (FIG. 5B) ESI MS/MS spectrum consistent with one of thymosin β4's tryptic peptides, [TETQEKNPLPSK]20-31.

FIG. 6—Thymosin β4 intensity levels based on MS signal intensities in the three different glomerular groups. Statistical significance of the differences was determined by using a two-tailed Student's t test for normal vs. nonsclerotic glomeruli (p=0.015), normal vs. sclerotic glomeruli (p<0.05), and nonsclerotic vs. sclerotic glomeruli (p=0.4) comparisons. The error bars correspond to 95% confidence intervals.

FIG. 7—Thymosin β4 (brown) was not detectable in normal glomeruli (left), but was increased in sclerotic glomeruli (right). Staining was not present in macrophages (red), staining on serial sections with endothelial and mesangial markers (not shown) confirmed predominantly endothelial localization (anti-thymosin β4, brown; anti-CD68, red, 200×).

FIG. 8—Western blot of thymosin β4 expression in cultured glomerular endothelial cells and podocytes. Mouse muscle and spleen tissue were used as negative and positive controls, respectively.

FIG. 9—Top: Representative Western blot of replicate experiments of thymosin β4 and Ang II-induced PAI-1. GEN were stimulated with Ang II (10-6 M) for 24 hrs, with or without concomitant transfection with siRNA or control siRNA for thymosin β4. Normal GEN at baseline expressed thymosin β4 (lane 1), which was successfully knocked down about 90% using siRNA (lane 2 and 5 versus lane 3). Neither siRNA nor control siRNA affected baseline PAI-1 expression (lane 1 vs lane 2 and lane 3). Ang II dramatically upregulated PAI-1 in normal GEN (lane 4). Transfection with siRNA markedly dampened this Ang II induction of PAI-1 (lane 5). Control siRNA had no effect (lane 6). Bottom: Average PAI-1 protein expression of replicate Western blot experiments, normalized to β-actin.

### DESCRIPTION OF ILLUSTRATIVE EMBODIMENTS

The present inventors have now demonstrated that LCM and MALDI-TOF MS can accurately classify the proteomic profiles of normal versus nonsclerotic versus sclerotic glomeruli. Using overall proteomic pattern similarity comparisons, non-sclerotic glomeruli were found to have more similarities to the sclerotic glomeruli than to the normal glomeruli. Thymosin β4 was identified as one of the key differentially expressed protein markers upregulated both in nonsclerotic and sclerotic glomeruli in the ⅚ nephrectomy model. Immunohistochemistry confirmed thymosin β4 elevated expression levels in sclerotic glomeruli, localized particularly to endothelial cells. Using RNAi technology, thymosin β4 was found to regulate a key pathway in sclerosis, namely angiotensin II-induced PAI-1 expression.

Normal and sclerotic glomeruli represent the two extremes of evolution of glomerulosclerosis. In this study, the inventors determined that proteomic patterns for these normal and sclerotic glomeruli are markedly different. Of greater interest is the finding that non-sclerotic glomeruli in the setting of progressive sclerosis also have an altered proteomic profile, more closely related to sclerotic than normal glomeruli. Foot process effacement occurs in all glomeruli in FSGS, regardless of whether sclerosis is present in the glomerulus or not. Thus, podocyte-related molecules would be expected to be altered in both nonsclerotic and sclerotic glomeruli compared to normal baseline. It is likely that such additional proteins, perhaps of higher molecular weights, not detected by the LCM-MALDI MS approach, would also be differentially upregulated in both non-sclerotic and sclerotic glomeruli versus normal.

However, podocyte effacement does not per se correlate with progressive sclerosis. In human disease, both FSGS and minimal change disease (MCD) are characterized by diffuse foot process effacement. Non-sclerosis develops in MCD, and prognosis is excellent, in contrast to the progressive scarring of FSGS (Fogo et al., 1990). In small human renal biopsies, it may be difficult to differentiate between MCD versus FSGS, if the defining segmental sclerotic lesion is not sampled. Differential downregulation of GBM α- and β-dystroglycan by immunostaining in MCD, but not FSGS, has been proposed as a tool for distinction of these two entities (Good et al., 2004). Thus, the inventors hypothesize that increased thymosin β4 may be an additional such marker to allow identification of early sclerotic processes.

The data presented here further indicate that the non-sclerotic glomeruli in FSGS not only have foot process effacement, but also have activation of proscierotic mechanisms. This has important implications for therapy, as it suggests that these glomeruli are further activated towards sclerosis than standard light microscopy and electron microscopy would suggest, and more targeted, aggressive therapy might be needed to effect remission or even regression of sclerosis (Fogo et al., 2000).

The inventors postulated that identification of the differentially expressed protein markers in the early, prosclerotic stage of progression could advance the understanding of pathomechanisms and possibly direct such therapies. They found, by proteomic analysis, that thymosin β4 expression level was elevated in sclerotic and nonsclerotic glomeruli vs baseline. This upregulation of thymosin β4 was further confirmed to be localized to endothelial cells by immunohistochemistry in vivo and cell culture analyses. Thymosin β4 is a highly conserved protein which has a wide range of functions. Thymosin β4 is an intracellular G-actin sequestering proteins (Safer et al., 1990), and plays a role in wound healing, angiogenesis, and endothelial cell differentiation (Grant et al., 1999). Recently, thymosin β4 was reported to increase plasminogen activator inhibitor 1 (PAI-1) expression at both the mRNA and protein levels in endothelial cells (Al-Nedawi et al., 1999). PAI-1 inhibits tissue-type plasminogen activator (t-PA) and urokinase-type plasminogen activator (u-PA), preventing the activation of plasminogen to plasmin, which further degrades not only fibrin, but also extracellular matrix (ECM) (Eddy, 2002). PAI-1 also inhibits u-PA-induced matrix metalloprotease-2, thus further inhibiting ECM degradation. PAI-1 is induced by angiotensin II in vivo and in vitro, and is tightly linked to glomerulosclerosis (Ma et al., 2001). Conversely, PAI-1 downregulation is linked to amelioration or even regression of glomerulosclerosis (Fogo et al., 2000). Therefore, upregulation of thymosin β4 could promote the glomerulosclerosis process via upregulation of PAI-1.

To further explore the role of thymosin β4 in this sclerotic pathway, the inventors modulated its expression in cultured endothelial cells. Downregulation of thymosin β4 by RNAi technology decreased angiotensin II-induced PAI-1 expression. These findings imply that thymosin β4, via effects on PAI-1, is not only a marker, but potentially a contributor, to glomerulosclerosis.

In conclusion, using laser capture microdissection combined with MALDI mass spectrometry technology, specific proteomic patterns were obtained that accurately classified normal vs non-sclerotic or sclerotic glomeruli in FSGS. The proteomic pattern of nonsclerotic glomeruli in a fibrosing kidney was found to be more similar to the proteomic pattern of sclerotic glomeruli than to normal glomeruli, suggesting that non-sclerotic glomeruli have early activation of prosclerotic mechanisms. As a discovery tool, our proteomic study further found thymosin β4 to be a key protein marker of glomerulosclerosis and possibly even a contributor to progression by promoting angiotensin-induced PAI-1 expression.

I. Glomeruli and Glomerulosclerosis

The glomerulus is a capillary bed found surrounded by the Bowman's capsule of the nephron in the vertebrate kidney. Glomerular endothelial cells are perforated by thousands of small cracks known as fenestrae. These cracks allow water and small solutes to pass through, but not proteins and cells. Blood is fed to the glomerulus through the afferent arteriole, and empties into the efferent arteriole. The difference in pressure in the arterioles results in the process of ultrafiltration where fluids and soluble materials in the blood are forced out of the capillaries and into the Bowman's space. Fluids collected in the Bowman's space is known as glomerular filtrate, which eventually becomes urine after further processing along the nephron.

1. Glomerulosclerosis

Glomerulosclerosis is the scarring of kidney blood vessels—glomeruli—which are the functional units in the kidney that filter urine from the blood. A sign of glomerulosclerosis is proteinuria, as the scarring disturbs the filtering process and allows protein to leak from the blood into the urine.

But glomerulosclerosis is just one of many possible causes of proteinuria. To find out whether a patient has glomerulosclerosis or some other kidney problem, the doctor often performs a kidney biopsy using a special needle to remove a tiny sample of the kidney to be examined under a microscope. About 15 percent of people with proteinuria turn out to have glomerulosclerosis.

Glomerulosclerosis can develop in children and adults and may result from different types of kidney conditions. One kind of glomerulosclerosis frequently encountered is caused by diabetes. Focal segmental glomerulosclerosis (FSGS), another chronic kidney condition, may be caused by infection or drug use and it may occur in patients with AIDS. However, most cases of FSGS are of unknown cause.

The early stages of glomerular disease may not cause any symptoms. As discussed above, one of the most important warning signs of glomerular disease is proteinuria, usually discovered during a routine medical exam. The loss of large amounts of protein may cause swelling in the ankles or accumulation of fluid in the abdomen. Scarred glomeruli cannot be repaired. Many patients with glomerulosclerosis gradually get worse until their kidneys fail completely. This condition is called end-stage renal disease or ESRD. Patients with ESRD must go on dialysis (hemodialysis or peritoneal dialysis) to clean their blood or get a new kidney through transplantation. A patient who has just received a diagnosis of glomerulosclerosis may reach ESRD within a variable period of time; it can be a year, or it may take 10 years or more.

The best treatment for glomerulosclerosis depends upon what caused the scarring, which can be determined by renal biopsy. Immunosuppressants—drugs that block the body's immune system—stop proteinuria in about half of the patients with glomerulosclerosis. But when the course of treatment is over, proteinuria returns for many patients. In some cases, the drugs actually may end up hurting the kidneys of certain patients.

Most doctors try to slow down the progression of kidney failure by controlling the patient's blood pressure. A class of blood pressure medicines called ACE inhibitors appears to preserve kidney function in patients with diabetes. Further studies may show that ACE inhibitors slow down kidney failure even in patients who do not have diabetes. Some doctors advise their patients to go on a low-protein diet to lighten the load of wastes on the kidneys. Some kidney patients may need to control their cholesterol through diet or both diet and medicine.

2. Focal Segmental Glomerulosclerosis

Focal Segmental Glomerulosclerosis (FSGS), like other forms of glomerulosclerosis, is a kidney disorder involving formation of scar tissue in some of the glomeruli. The cause of focal segmental glomerulosclerosis is usually unknown. A minority of cases result from reflux nephropathy. Some (but not all) of the glomeruli become scarred. It affects about 1 out of 10,000 people, both children and adults. Men are affected slightly more often than women, and it can occur in children. The main result of focal segmental glomerulosclerosis is nephrotic syndrome, and FSGS is responsible for about 10 to 15% of all cases of nephrotic syndrome. Protein is persistently excreted in the urine, especially urine albumin. Most cases will progress to chronic renal failure.

Although the disorder seems to be immune-system related, response to corticosteroid or immunosuppressive medications is inconsistent. Symptoms include foamy urine, swelling of the body (generalized edema from retained fluids), weight gain and poor appetite. Signs of chronic renal failure and associated fluid overload may develop as the disorder progresses. Tests may include a urinalysis, which shows protein, with or without small amounts of blood. A renal biopsy, which shows scarring of parts of a glomerulus (focal) or of only some of the glomeruli (segmental).

The goal of treatment is control of the symptoms associated with nephrotic syndrome and chronic renal failure. Treatment may be chronic and lifelong. Corticosteroids and immunosuppressive medications may be prescribed to reduce the immune response. Antihypertensive and diuretic medications may help control symptoms such as high blood pressure and edema. Antibiotics may be needed to control infections.

The treatment of high blood cholesterol and triglyceride levels, which are also common with this disorder, may be recommended to reduce the development of atherosclerosis. Dietary limitation of cholesterol and saturated fats may be of only limited benefit as the high levels seem to result from overproduction of cholesterol and triglycerides by the liver rather than the excessive intake of fats. Nonetheless, medications to reduce cholesterol and triglycerides may be recommended.

High-protein diets are of debatable value. In many patients, reducing the amount of protein in diet produces a decrease in urine protein. In most cases, a moderate-protein diet (1 gram of protein per kilogram of body weight per day) is usually recommended. In cases in which renal failure is present, a low-protein diet may be preferred. The sodium (salt) in the diet and/or fluids may be restricted to help control swelling. Vitamin D may need to be replaced if nephrotic syndrome is chronic and unresponsive to therapy.

Ultimately, dialysis or kidney transplantation may be necessary to control renal failure in late stage disease. This is common, as over 50% of focal or segmental glomerulosclerosis cases develop chronic renal failure within 10 years.

II. FSGS-Related Genes and Their Classification

As discussed above, the present invention provides a protein-based grading of glomerular tissue base. At present, this classification is based on the identification of five proteins species, four identified only by molecular weight, the expression of which correlates with the various sclerotic states. Using information derived from these four targets, one can classify tissue as normal, non-sclerotic or sclerotic.

1. Thymosin β4

Thymosin β4 has a Mr of 4982 and an isoelectric point of 5.1. The complete amino acid sequence of this polypeptide has been established by automated Edman degradation as well as by manual sequence analysis, and is composed of 43 amino acid residues with acetylserine at the NH₂ terminus. This molecule induces expression of terminal deoxynucleotidyl transferase in transferase-negative murine thymocytes in vivo and in vitro. It also exhibits ability to inhibit the migration of macrophages. Comparison of the sequence of thymosin β4 to other thymic hormones or other published protein sequences does not reveal any statistically significant relationship. Two helical regions were identified in the structure using data for prediction of protein conformation (Low & Goldstein, 1982).

Thymosin beta 4 sulfoxide is generated by monocytes in the presence of glucocorticoids and acts as a signal to inhibit an inflammatory response. In vitro, thymosin beta 4 sulfoxide inhibited neutrophil chemotaxis, and in vivo, the oxidized peptide, but not the native form, was a potent inhibitor of carrageenin-induced edema in the mouse paw. Thymosin β4 is unique in that oxidation attenuates its intracellular G-actin sequestering activity, but greatly enhances its extracellular signaling properties (Young et al. 1999). Addition of thymosin β4 topically or intraperitoneally increases reepithelialization by 42% over saline controls at 4 d and by as much as 61% at 7 d post-wounding. Treated wounds also contracted at least 11% more than controls by day 7. Increased collagen deposition and angiogenesis are observed in the treated wounds (Malinda et al., 1999).

Thymosin β4 has been found to bind actin in human platelet extracts and to inhibit actin polymerization in vitro, raising the possibility that it may be a physiological regulator of actin assembly. To examine this potential function, cellular beta 4 concentration was increased by microinjecting synthetic beta 4 into living epithelial cells and fibroblasts. The injection induced a diminution of stress fibers and a dose-dependent depolymerization of actin filaments as indicated by quantitative image analysis of phalloidin binding. These results show that thymosin β4 is a potent regulator of actin assembly in living cells (Sanders et al., 1992).

2. Other Markers

The present inventors have identified four additional protein, having molecular weights of 4222 Daltons, 5485 Daltons, 7018 Daltons and 12,131 Daltons, which are useful in discriminating normal, non-sclerotic and sclerotic tissue. While the identify of these proteins remains to be elucidated, they may nonetheless be used to characterize the tissues in question.

III. Protein-Based Detection—Immunodetection

Thus, in accordance with the present invention, methods are provided for the assaying of protein expression in patients suspected of having or at risk of developing glomerulosclerosis. As discussed above, the principal applications of this assay are to distinguish between normal, non-sclerotic and sclerotic tissues. In each of these assays, the expression of a particular set of target proteins, set forth in the preceding sections, will be measured.

There are a variety of methods that can be used to assess protein expression. One such approach is to perform protein identification with the use of antibodies. As used herein, the term “antibody” is intended to refer broadly to any immunologic binding agent such as IgG, IgM, IgA, IgD and IgE. Generally, IgG and/or IgM are preferred because they are the most common antibodies in the physiological situation and because they are most easily made in a laboratory setting. The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art. Means for preparing and characterizing antibodies, both polyclonal and monoclonal, are also well known in the art (see, e.g., Antibodies: A Laboratory Manual, Cold Spring Harbor Laboratory, 1988; incorporated herein by reference). In particular, antibodies to calcyclin, calpactin I light chain, astrocytic phosphoprotein PEA-15 and tubulin-specific chaperone A are contemplated.

In accordance with the present invention, immunodetection methods are provided. Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. The steps of various useful immunodetection methods have been described in the scientific literature, such as, e.g., Doolittle & Ben-Zeev O, 1999; Gulbis & Galand, 1993; De Jager et al., 1993; and Nakamura et al., 1987, each incorporated herein by reference.

In general, the immunobinding methods include obtaining a sample suspected of containing a relevant polypeptide, and contacting the sample with a first antibody under conditions effective to allow the formation of immunocomplexes. In terms of antigen detection, the biological sample analyzed may be any sample that is suspected of containing an antigen, such as, for example, a tissue section or specimen, a homogenized tissue extract, a cell, or even a biological fluid.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any antigens present. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection designed by Charles Cantor uses two different antibodies. A first step biotinylated, monoclonal or polyclonal antibody is used to detect the target antigen(s), and a second step antibody is then used to detect the biotin attached to the complexed biotin. In that method the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

As detailed above, immunoassays are in essence binding assays. Certain immunoassays are the various types of enzyme linked immunosorbent assays (ELISAs) and radioimmunoassays (RIA) known in the art. However, it will be readily appreciated that detection is not limited to such techniques, and Western blotting, dot blotting, FACS analyses, and the like may also be used.

In one exemplary ELISA, the antibodies of the invention are immobilized onto a selected surface exhibiting protein affinity, such as a well in a polystyrene microtiter plate. Then, a test composition suspected of containing the antigen, such as a clinical sample, is added to the wells. After binding and washing to remove non-specifically bound immune complexes, the bound antigen may be detected. Detection is generally achieved by the addition of another antibody that is linked to a detectable label. This type of ELISA is a simple “sandwich ELISA.” Detection may also be achieved by the addition of a second antibody, followed by the addition of a third antibody that has binding affinity for the second antibody, with the third antibody being linked to a detectable label.

In another exemplary ELISA, the samples suspected of containing the antigen are immobilized onto the well surface and then contacted with the anti-ORF message and anti-ORF translated product antibodies of the invention. After binding and washing to remove non-specifically bound immune complexes, the bound anti-ORF message and anti-ORF translated product antibodies are detected. Where the initial anti-ORF message and anti-ORF translated product antibodies are linked to a detectable label, the immune complexes may be detected directly. Again, the immune complexes may be detected using a second antibody that has binding affinity for the first anti-ORF message and anti-ORF translated product antibody, with the second antibody being linked to a detectable label.

Another ELISA in which the antigens are immobilized, involves the use of antibody competition in the detection. In this ELISA, labeled antibodies against an antigen are added to the wells, allowed to bind, and detected by means of their label. The amount of an antigen in an unknown sample is then determined by mixing the sample with the labeled antibodies against the antigen during incubation with coated wells. The presence of an antigen in the sample acts to reduce the amount of antibody against the antigen available for binding to the well and thus reduces the ultimate signal. This is also appropriate for detecting antibodies against an antigen in an unknown sample, where the unlabeled antibodies bind to the antigen-coated wells and also reduces the amount of antigen available to bind the labeled antibodies.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background. The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

The antibodies of the present invention may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors, and/or is well known to those of skill in the art (Brown et al., 1990; Abbondanzo et al., 1999; Allred et al., 1990).

Also contemplated in the present invention is the use of immunohistochemistry. This approach uses antibodies to detect and quantify antigens in intact tissue samples. Generally, frozen-sections are prepared by rehydrating frozen “pulverized” tissue at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and cutting up to 50 serial permanent sections.

IV. Protein-Based Detection—Mass Spectromety

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) (Chen et al., 2001; Zhong et al., 2001; Wu et al., 2000) while newer quantitative methods are being developed using matrix assisted laser desorption/ionization (MALDI) followed by time of flight (TOF) MS (Bucknall et al., 2002; Mirgorodskaya et al., 2000; Gobom et al., 2000). In accordance with the present invention, one can generate mass spectrometry profiles that are useful for discriminating normal, non-sclerotic and sclerotic tissue. In particular, one may examine thymosin β4 expression in target tissues.

1. ESI

ESI is a convenient ionization technique developed by Fenn and colleagues (Fenn et al., 1989) that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

A typical conventional ESI source consists of a metal capillary of typically 0.1-0.3 mm in diameter, with a tip held approximately 0.5 to 5 cm (but more usually 1 to 3 cm) away from an electrically grounded circular interface having at its center the sampling orifice, such as described by Kabarle et al. (1993). A potential difference of between 1 to 5 kV (but more typically 2 to 3 kV) is applied to the capillary by power supply to generate a high electrostatic field (10⁶ to 10⁷ V/m) at the capillary tip. A sample liquid carrying the analyte to be analyzed by the mass spectrometer, is delivered to tip through an internal passage from a suitable source (such as from a chromatograph or directly from a sample solution via a liquid flow controller). By applying pressure to the sample in the capillary, the liquid leaves the capillary tip as a small highly electrically charged droplets and further undergoes desolvation and breakdown to form single or multicharged gas phase ions in the form of an ion beam. The ions are then collected by the grounded (or negatively charged) interface plate and led through an the orifice into an analyzer of the mass spectrometer. During this operation, the voltage applied to the capillary is held constant. Aspects of construction of ESI sources are described, for example, in U.S. Pat. Nos. 5,838,002; 5,788,166; 5,757,994; RE 35,413; and 5,986,258.

2. ESI/MS/MS

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals (Zweigenbaum et al., 2000; Zweigenbaum et al., 1999) and bioactive peptides (Desiderio et al., 1996; Lovelace et al., 1991). Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide (Duncan et al., 1993; Bucknall et al., 2002). Protein quantification has been achieved by quantifying tryptic peptides (Mirgorodskaya et al., 2000). Complex mixtures such as crude extracts can be analyzed, but in some instances sample clean up is required (Nelson et al., 1994; Gobom et al., 2000).

3. SIMS

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

4. LD-MS and LDLPMS Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer, and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments are due to different size causing different velocity. Each ion mass will thus have a different flight-time to the detector.

One can either form positive ions or negative ions for analysis. Positive ions are made from regular direct photoionization, but negative ion formation require a higher powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectra.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

5. MALDI-TOF-MS

Since its inception and commercial availability, the versatility of MALDI-TOF-MS has been demonstrated convincingly by its extensive use for qualitative analysis. For example, MALDI-TOF-MS has been employed for the characterization of synthetic polymers (Marie et al., 2000; Wu et al., 1998). peptide and protein analysis (Zuluzec et al., 1995; Roepstorff et al., 2000; Nguyen et al., 1995), DNA and oligonucleotide sequencing (Miketova et al., 1997; Faulstich et al., 1997; Bentzley et al., 1996), and the characterization of recombinant proteins (Kanazawa et al., 1999; Villanueva et al., 1999). Recently, applications of MALDI-TOF-MS have been extended to include the direct analysis of biological tissues and single cell organisms with the aim of characterizing endogenous peptide and protein constituents (Li et al., 2000; Lynn et al., 1999; Stoeckli et al., 2001; Caprioli et al., 1997; Chaurand et al., 1999; Jespersen et al., 1999).

The properties that make MALDI-TOF-MS a popular qualitative tool-its ability to analyze molecules across an extensive mass range, high sensitivity, minimal sample preparation and rapid analysis times—also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use (Kazmaier et al., 1998; Horak et al., 2001; Gobom et al., 2000; Wang et al., 2000; Desiderio et al., 2000). These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products (Wang et al., 1999; Jiang et al., 2000; Wang et al., 2000; Yang et al., 2000; Wittmann et al., 2001), whereas other studies have demonstrated the potential of MALDI-TOF-MS for the quantification of biologically relevant analytes such as neuropeptides, proteins, antibiotics, or various metabolites in biological tissue or fluid (Muddiman et al., 1996; Nelson et al., 1994; Duncan et al., 1993; Gobom et al., 2000; Wu et al., 1997; Mirgorodskaya et al., 2000). In earlier work it was shown that linear calibration curves could be generated by MALDI-TOF-MS provided that an appropriate internal standard was employed (Duncan et al., 1993). This standard can “correct” for both sample-to-sample and shot-to-shot variability. Stable isotope labeled internal standards (isotopomers) give the best result.

With the marked improvement in resolution available on modern commercial instruments, primarily because of delayed extraction (Bahr et al., 1997; Takach et al., 1997), the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated, but are not used routinely.

V. Nucleic Acid Detection

In alternative embodiments for detecting protein expression, one may assay for gene transcription. For example, an indirect method for detecting protein expression is to detect mRNA transcripts from which the proteins are made. The following is a discussion of such methods, which are applicable, particularly to thymosin β4, in the context of the present invention.

1. Hybridization

There are a variety of ways by which one can assess gene expression. These methods either look at protein or at mRNA levels. Methods looking at mRNAs all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary, but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the specification are incorporated herein by reference.

2. Amplification of Nucleic Acids

Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid under conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously (Chamberlan et al., 1990). The presence of many PCR primers in a single tube could cause many problems, such as the increased formation of misprimed PCR products and “primer dimers,” the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller & Wold (1989).

Another method for amplification is ligase chain reaction (“LCR”), disclosed in European Application No. 320 308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al., 1989).

3. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be effected by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 1989). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

4. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed, and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

VI. Gene Therapy

In another embodiment, the present invention provides for the administration of a gene therapy vector encoding one or more genes identified as being down-regulated in glomerulosclerosis. Alternatively, for genes that are overexpressed in glomerulsclerosis, the transgenes may provide for reduced expression of appropriate targets. Various aspects of gene delivery and expression are set forth below.

1. Therapeutic Transgenes

Thus, in accordance with the present invention, there are provided methods of treating cancer utilizing genes identified as being overexpressed or underexpressed in glomerulosclerosis. By inhibiting or increasing the expression of various of these genes, therapeutic benefit may be provided to patients.

2. Antisense

The term “antisense” nucleic acid refers to oligo- and polynucleotides complementary to bases sequences of a target DNA or RNA. When introduced into a cell, antisense molecules hybridize to a target nucleic acid and interfere with its transcription, transport, processing, splicing or translation. Targeting double-stranded DNA leads to triple helix formation; targeting RNA will lead to double helix formation.

Antisense constructs may be designed to bind to the promoter or other control regions, exons, introns or even exon-intron boundaries of a gene. Antisense RNA constructs, or DNA encoding such antisense RNAs, may be employed to inhibit gene transcription or translation within a host cell. Nucleic acid sequences which comprise “complementary nucleotides” are those which are capable of base-pairing according to the standard Watson-Crick complementarity rules. That is, that the larger purines will base pair with the smaller pyrimidines to form combinations of guanine paired with cytosine (G:C) and adenine paired with either thymine in the case of DNA (A:T), or uracil (A:U) in the case of RNA. Inclusion of less common bases such as inosine, 5-methylcytosine, 6-methyladenine, hypoxanthine and others in hybridizing sequences does not interfere with pairing.

As used herein, the terms “complementary” and “antisense sequences” mean nucleic acid sequences that are substantially complementary over their entire length and have very few base mismatches. For example, nucleic acid sequences of fifteen bases in length may be termed complementary when they have complementary nucleotides at thirteen or fourteen positions. Naturally, nucleic acid sequences with are “completely complementary” will be nucleic acid sequences which have perfect base pair matching with the target sequences, i.e., no mismatches. Other sequences with lower degrees of homology are contemplated. For example, an antisense construct with limited regions of high homology, but overall containing a lower degree (50% or less) total homology, may be used.

While all or part of the gene sequence may be employed in the context of antisense construction, statistically, any sequence of 17 bases long should occur only once in the human genome and, therefore, suffice to specify a unique target. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that oligonucleotides of 8, 9, 10, 11, 12, 13, 14, 15, 16, 17, 18, 19, 20 or more base pairs will be used. One can readily determine whether a given antisense nucleic acid is effective at targeting a gene simply by testing the construct in vitro to determine whether the gene's function or expression is affected.

In certain embodiments, one may wish to employ antisense constructs which include other elements, for example, those which include C-5 propyne pyrimidines. Oligonucleotides which contain C-5 propyne analogs of uridine and cytidine have been shown to bind RNA with high affinity and to be potent inhibitors or gene expression. Wagner et al. (1993).

3. Ribozymes

The term “ribozyme” refers to an RNA-based enzyme capable of targeting and cleaving particular DNA and RNA sequences. Ribozymes can either be targeted directly to cells, in the form of RNA oligonucleotides incorporating ribozyme sequences, or introduced into the cell as an expression construct encoding the desired ribozymal RNA. Ribozymes may be used and applied in much the same way as described for antisense nucleic acids. Ribozyme sequences also may be modified in much the same way as described for antisense nucleic acids. For example, one could include modified bases or modified phosphate backbones to improve stability or function.

4. Single Chain Antibodies

Naturally-occurring antibodies (of isotype IgG) produced by B cells, consist of four polypeptide chains. Two heavy chains (composed of four immunoglobulin domains) and two light chains (made up of two immunoglobulin domains) are held together by disulphide bonds. The bulk of the antibody complex is made up of constant immunoglobulin domains. These have a conserved amino acid sequence, and exhibit low variability. Different classes of constant regions in the stem of the antibody generate different isotypes of antibody with differing properties. The recognition properties of the antibody are carried by the variable regions (VH and VL) at the ends of the arms. Each variable domain contains three hypervariable regions known as complementarity determining regions, or CDRs. The CDRs come together in the final tertiary structure to form an antigen binding pocket. The human genome contains multiple fragments encoding portions of the variable domains in regions of the immunoglobulin gene cluster known as V, D and J. During B cell development these regions undergo recombination to generate a broad diversity of antibody affinities. As these B cell populations mature in the presence of a target antigen, hypermutation of the variable region takes place, with the B cells producing the most active antibodies being selected for further expansion in a process known as affinity maturation.

A major breakthrough was the generation of monoclonal antibodies, pure populations of antibodies with the same affinity. This was achieved by fusing B cells taken from immunized animals with myeloma cells. This generates a population of immortal hybridomas, from which the required clones can be selected. Monoclonal antibodies are very important research tools, and have been used in some therapies. However, they are very expensive and difficult to produce, and if used in a therapeutic context, can elicit and immune response which will destroy the antibody. This can be reduced in part by humanizing the antibody by grafting the CDRs from the parent monoclonal into the backbone of a human IgG antibody. It may be better to deliver antibodies by gene therapy, as this would hopefully provide a constant localized supply of antibody following a single dose of vector. The problems of vector design and delivery are dealt with elsewhere, but antibodies in their native form, consisting of two different polypeptide chains which need to be generated in approximately equal amounts and assembled correctly are not good candidates for gene therapy. However, it is possible to create a single polypeptide which can retain the antigen binding properties of a monoclonal antibody.

The variable regions from the heavy and light chains (VH and VL) are both approximately 110 amino acids long. They can be linked by a 15 amino acid linker (e.g., (glycine₄serine)₃), which has sufficient flexibility to allow the two domains to assemble a functional antigen binding pocket. Addition of various signal sequences allows the scFv to be targeted to different organelles within the cell, or to be secreted. Addition of the light chain constant region (Ck) allows dimerization via disulphide bonds, giving increased stability and avidity. However, there is evidence that scFvs spontaneously multimerize, with the extent of aggregation (presumably via exposed hydrophobic surfaces) being dependent on the length of the glycine-serine linker.

The variable regions for constructing the scFv are obtained as follows. Using a monoclonal antibody against the target of interest, it is a simple procedure to use RT-PCR to clone out the variable regions from mRNA extracted from the parent hybridoma. Degenerate primers targeted to the relatively invariant framework regions can be used. Expression constructs are available with convenient cloning sites for the insertion of the cloned variable regions.

5. siRNA

RNA interference (also referred to as “RNA-mediated interference” or RNAi) is a mechanism by which gene expression can be reduced or eliminated. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin and Avery et al., 1999; Montgomery et al., 1998; Sharp and Zamore, 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin and Avery et al., 1999; Montgomery et al., 1998; Sharp et al., 1999; Sharp and Zamore, 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp et al., 1999; Sharp and Zamore, 2000; Elbashir et al., 2001). It is generally accepted that RNAi acts post-transcriptionally, targeting RNA transcripts for degradation. It appears that both nuclear and cytoplasmic RNA can be targeted (Bosher and Labouesse, 2000).

siRNAs must be designed so that they are specific and effective in suppressing the expression of the genes of interest. Methods of selecting the target sequences, i.e., those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double-stranded RNA. Methods of chemical synthesis are diverse. Non-limiting examples are provided in U.S. Pat. Nos. 5,889,136, 4,415,723, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM, but concentrations of about 100 nM have achieved effective suppression of expression in mammalian cells. siRNAs have been most effective in mammalian cell culture at about 100 nM. In several instances, however, lower concentrations of chemically synthesized siRNA have been used (Caplen, et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

6. Vectors

In accordance with the present invention, both stimulatory and inhibitory genes may be provided to a cancer cell and expressed therein. Stimulatory genes are generally simply copies of the gene of interest, although in some cases they may be genes, the expression of which direct the expression of the gene of interest. Inhibitory genes, discussed above, may include expression constructs for antisense molecules, ribozymes, interfering RNAs or single-chain antibodies.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1989 and Ausubel et al., 1994, both incorporated herein by reference).

The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions as well and are described infra.

a. Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 1 lists non-limiting examples of elements/promoters that may be employed, in the context of the present invention, to regulate the expression of a RNA. Table 2 provides non-limiting examples of inducible elements, which are regions of a nucleic acid sequence that can be activated in response to a specific stimulus.

TABLE 1
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell and/or Villarreal., 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | Poly(rI)x | Tavernier et al., 1983 |
| | Poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | ElA, SV40 Large T | Taylor et al., 1989, 1990a, 1990b |
| | Antigen | |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

b. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′-methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

c. Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

d. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

e. Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

f. Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

g. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

h. Selectable and Screenable Markers

In certain embodiments of the invention, cells containing a nucleic acid construct of the resent invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is calorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

i. Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

j. Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells (e.g., mammalian cells). Non-limiting examples of virus vectors that may be used to deliver a nucleic acid of the present invention are described below.

i. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz, 1992).

ii. AAV Vectors

The nucleic acid may be introduced into the cell using adenovirus assisted transfection. Increased transfection efficiencies have been reported in cell systems using adenovirus coupled systems (Kelleher and Vos, 1994; Cotten et al., 1992; Curiel, 1994). Adeno-associated virus (AAV) is an attractive vector system as it has a high frequency of integration and it can infect non-dividing cells, thus making it useful for delivery of genes into mammalian cells, for example, in tissue culture (Muzyczka, 1992) or in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. Nos. 5,139,941 and 4,797,368, each incorporated herein by reference.

iii. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

In order to construct a retroviral vector, a nucleic acid (e.g., one encoding gene of interest) is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

iv. Other Viral Vectors

Other viral vectors may be employed as vaccine constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

V. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

7. Vector Delivery and Cell Transformation

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989; Nabel and Baltimore, 1987), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al., 1986; Potter et al., 1984); by calcium phosphate precipitation (Graham and Van Der Eb, 1973; Chen and Okayama, 1987; Rippe et al., 1990); by using DEAE-dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al., 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

a. Injection

In certain embodiments, a nucleic acid may be delivered to a glomerulus or kidney via one or more injections (i.e., a needle injection). Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub, 1985).

b. DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal, 1985).

c. Sonication Loading

Additional embodiments of the present invention include the introduction of a nucleic acid by direct sonic loading. LTK⁻ fibroblasts have been transfected with the thymidine kinase gene by sonication loading (Fechheimer et al., 1987).

d. Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene, 1982; Fraley et al., 1979; Nicolau et al., 1987). The feasibility of liposome-mediated delivery and expression of foreign DNA in cultured chick embryo, HeLa and hepatoma cells has also been demonstrated (Wong et al., 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al., 1989). In other embodiments, a liposome may be complexed or employed in conjunction with nuclear non-histone chromosomal proteins (HMG-1) (Kato et al., 1991). In yet further embodiments, a liposome may be complexed or employed in conjunction with both HVJ and HMG-1. In other embodiments, a delivery vehicle may comprise a ligand and a liposome.

e. Receptor-Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al., 1987). It is contemplated that the tissue-specific transforming constructs of the present invention can be specifically delivered into a target cell in a similar manner.

VII. Pharmaceutical Formulations and Routes of Administration

Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals.

The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Supplementary active ingredients also can be incorporated into the compositions.

Administration of these compositions according to the present invention will be via any common route so long as the target tissue is available via that route. This includes intradermal, subcutaneous, intramuscular, intraperitoneal or intravenous injection. In particular, intratumoral routes and sites local and regional to tumors are contemplated. Such compositions would normally be administered as pharmaceutically acceptable compositions, described supra.

The active compounds also may be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy administration by a syringe is possible. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

For oral administration the polypeptides of the present invention may be incorporated with excipients that may include water, binders, abrasives, flavoring agents, foaming agents, and humectants.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Materials and Methods

Remnant Rat Kidney Model. Adult male Sprague Dawley rats (n=6, 250-300 g, Charles River, Tenn., USA) were studied. Rats were housed under normal conditions with a 12-hr light/dark cycle, at 70° F. with 40% humidity and 12-air exchanges/hr and received normal rat chow and water ad lib (“5001” diet, Purina Laboratory Rodent diet, 23.4% protein, 4.5% fat, 6.0% fiber, 0.40% sodium). Rats underwent ⅚ nephrectomy under pentobarbital anesthesia by right unilateral nephrectomy (Nx) and ligation of branches of the left renal artery, producing a total of ⅚ renal ablation. Focal segmental glomerulosclerosis was well developed by 12 weeks after ⅚ Nx, and rats were then sacrificed. Glomerulosclerosis was defined as collapse and/or obliteration of glomerular capillary tuft accompanied by hyaline material and/or increase of matrix (Ikoma et al., 1991). Nephrectomized right kidneys obtained at baseline were used as normal control. A limited number of nonsclerotic glomeruli with normal histology were present in the remnant kidneys.

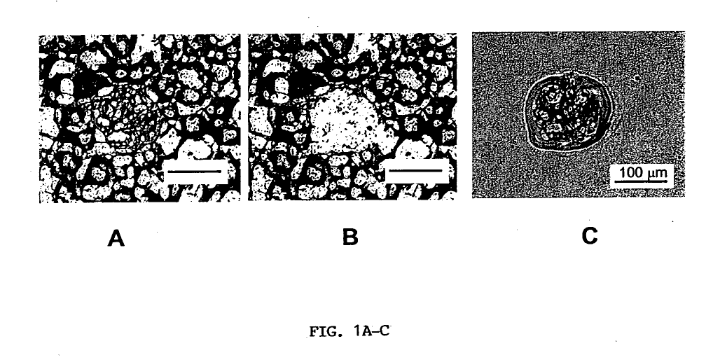

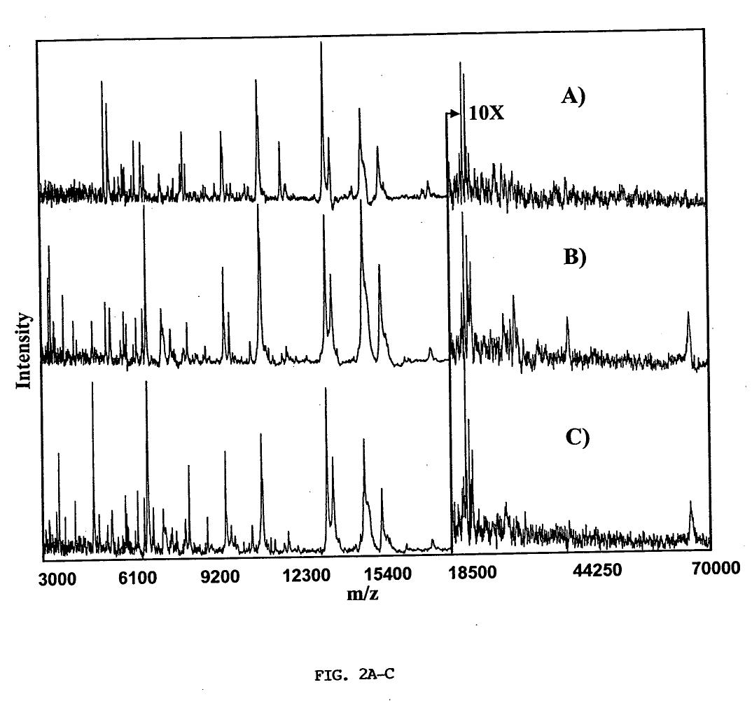

Laser Capture Microdissection. Protein profiles were obtained directly from laser capture microdissected cells using MALDI MS (Xu et al., 2002). Frozen kidney samples from ⅚ Nx or normal baseline were partially embedded in OCT and 5 μm thick cryostat sections were cut. The tissue sections were mounted on regular glass slides and dehydrated as follows: 70% ethanol 30s, 95% ethanol 1 min, 100% ethanol 1 min (2 times), xylene 2.5 min (2 times), and air dried. Rat kidney glomeruli were easily identified and classified as normal versus sclerotic. LCM allowed precise dissection of only glomeruli without surrounding tissue under LCM phase microscopy from surrounding cortex under LCM microscope without staining, as shown in (FIG. 1). Glomeruli were identified by phase microscopy as 1) normal in baseline kidney, and as 2) sclerotic or 3) nonsclerotic in ⅚ Nx. Glomeruli from each category were then microdissected using the Arcturus PixCell II LCM system (Mountain View, Calif., USA) with a focused 30 μm laser beam. These three groups of glomeruli were captured separately onto different LCM caps (Mountain View, Calif., USA). On average 50 normal glomeruli were captured from the control right kidney obtained during nephrectomy for each rat. From the remnant kidneys 12 weeks after ⅚ nephrectomy, 30 non-sclerotic and 50 sclerotic glomeruli were obtained from each rat using LCM. Nonsclerotic glomeruli were obtained from 5 of the 6 rats because one rat did not have sufficient nonsclerotic glomeruli present in the ablated kidney. All 6 rats with ⅚ Nx had sufficient sclerotic glomeruli for LCM analysis.

MALDI Mass Spectrometry. Protein expression profiles can be directly obtained from laser microdissected cells using MALDI MS (Xu et al., 2002). The LCM thermoplastic films with captured cells were peeled off from the LCM cap using forceps and mounted on a MALDI target plate using a conductive double-sided tape (Digi-Key, Thief River Falls, Minn., USA). Sinapinic acid (Sigma, St. Louis, Mo., USA) solution (20 mg/ml, 50/50/0.3, v/v/v, acetonitrile/water/TFA,) was microspotted on the captured cells under microscope visualization using pulled fine glass capillaries. Upon solvent dehydration, the matrix and proteins co-crystallized together. MALDI MS analyses were performed in the linear mode under the optimized delayed extraction condition on an Applied Biosystems DE-STR Voyager mass spectrometer (Framingham, Mass., USA). The crystals was irradiated by a series of pulsed laser (λ=337 nm) in a high vacuum. The protein analytes were desorbed and ionized from the surface of the tissue forming predominately singly charged protonated ions of the form [M+H]⁺, where M was the protein molecular weight. The protein ions were accelerated in a constant electric field and subsequently separated in a time-of-flight (TOF) mass analyzer. The mass-to-charge (m/z) ratio of each protein ion was recorded at the detector. Each mass spectrum was obtained averaging signals from 250 consecutive laser shots from five microdissected glomeruli. After internal calibration, the mass spectra were baseline subtracted and normalized using the software developed in our laboratory prior to statistical analysis.

Protein Identification. Remaining frozen rat kidney cortex (114 mg) samples from the same samples investigated by LCM were homogenized in 500 μl of protein extraction buffer using an ice-chilled Duall glass homogenizer. The protein extraction buffer is composed of 0.25 M sucrose (J. T. Baker, Phillipsburg, N.J., USA), 0.01 M Tris-HCl (J. T. Baker) and 0.1 mM PMSF (Sigma). The homogenate was centrifuged according to the following sequence: 10 minutes at 680 g, 10 minutes at 10,000 g, and 1 hr at 100,000 g. The final supernatant was filtered using a Millipore Ultrafree-MS 30,000 NMWL centrifugal filter device (Bedford, Mass., USA). A volume of 150 μl filtrate was separated on a Vydac 259VHP5415 polymeric column (Hesperia, Calif., USA) at 40° C. using a Waters Alliance HPLC system (Milford, Mass., USA). Solvent A contained 0.1% TFA (Burdick and Jackson, Muskegon, Mich., USA) in water and solvent B contained 0.085% TFA in acetonitrile (Fisher Scientific, Fair Lawn, N.J., USA). A flow rate of 1 ml/min was used with a gradient started from 5% B for 5 min, then in 55 min to 60% B, then in 10 min to 95% B and hold at 95% B for 10 min. The fractions were collected every minute and then completely dried using Thermo Quest Savant Speedvac (Holbrook, N.Y., USA). Dried HPLC fractions were reconstituted in 10 μl of 5/5/0.3, v/v/v, acetonitrile/water/TFA and analyzed by MALDI-TOF MS.

The fractions containing target protein markers, as identified by statistical analysis (see below) from LCM samples were completely lyophilized again and reconstituted with 10 μl of 0.4M ammonium hydrogen carbonate (Sigma). These fractions were reduced with 5 μl of 45 mM dithiothreitol (Sigma) in incubation at 60° C. for 15 min, followed by alkylation with 5 μl of 100 mM iodoacetamide (Sigma) in the dark for 15 min. One microliter of 1 μg/μl sequencing-grade trypsin (Promega, Madison, Wis., USA) was added, and the digestion allowed proceeding for 4 hrs at 37° C.

The digested fractions were subjected to LC-MS/MS analysis using a ThermoFinnigan LTQ mass spectrometer (San Jose, Calif., USA). Two microliters of sample were loaded into a 100 μm i.d. self-packed micro-capillary reverse phase column packed with Monitor C18-Spherical Silica from Column Engineering Inc (Ontario, Calif., USA). The mobile phase A was 0.1% formic acid (EM Science, Darmstadt, Germany) in water and phase B was 0.1% formic acid in acetonitrile. The gradient for mobile phase B started at 0% for 3 min, to 5% in 2 min, to 50% in 45 min, and to 90% in 5 min. The flow rate at the source was 700 nl/min. The fragment ion mass spectra were used to search the National Center for Biotechnology Information (NCBI) rat protein database using the SEQUEST algorithm (Eng et al., 1994).

Immunohistochemistry. For immunostaining, remnant rat kidney tissue was fixed in 4% paraformaldehyde overnight at 4° C., routinely processed and embedded in paraffin. Four micrometer sections were treated with 3% hydrogen peroxidase for 10 min, Power block (BioGenex Laboratories, San Ramon, Calif.) for 45 mins, incubated with primary rabbit anti-thymosin β4 antibody (Biodesign, Saco, Me.) for 1 hour at 37° C., and rinsed twice with PBS. HRP conjugated-swine ant-rabbit antibody (Dako, Carpenteria, Calif.) was added and incubated for 45 mins at RT. After rinsing 3 times with PBS, diaminobenzidene was added as a chromagen. Slides were counterstained with hematoxylin.

Infiltrating macrophages were detected with double-staining for thymosin β4 and mouse monoclonal antibody to EDI (BioSource International, Camarillo, Calif.), a macrophage marker, followed by biotinylated goat anti-mouse IgG (BioGenex) for 30 min and alkaline phosphatase-streptavidin conjugate (BioGenex) for 30 min. Sections were developed with sigma fast red TR/Naphtol AS-MX for 5 min, then counterstained and cover-slipped. Negative controls omitting the primary antibody and using nonspecific immunoglobulin showed no staining. Positive control using rat spleen showed the expected distribution of thymosin β4 (Mora et al, 1997). Glomerular endothelial cells and mesangial cells were identified on serial sections by immunostaining with anti-RECA-1 antibody (Abcam, Cambridge, Mass.) and mouse anti-rat CD90 (Thy-1) antibody (BD Pharmingen, San Diego, Calif.), respectively.

Cell Culture. Glomerular endothelial cells (GEN), derived from SV40 mice (gift from Dr. Michael Madaio) were grown in 10% fetal bovine serum (FBS) which had been heat-inactivated at 56° C. for 1 hr, with DMEM:Ham F12 media (low glucose DMEM, 6 mM) in a 3:1 ratio, with L-glytamin 2 mM and HEPES 10 mM added (Akis et al., 2004). The cells were grown at 37° C., with 5% CO₂ under humid conditions in Corning flasks. The cells showed CD31 expression, confirming endothelial cell phenotype. Primary cultures of podocyte were performed as follows: rat kidneys were removed and the renal capsules were stripped using autoclaved surgical instruments. The cortex was isolated and minced into small pieces with razor blade, and glomeruli isolated by sieving (sieve pore size 180μ X2, 75μ X1). Glomeruli were then suspended in DMEM/Ham's F-12 (2:1) containing 0.2 um-filtered 3T3-L1 supernatant, 5% heated-inactivated FCS, ITS solution, and 100 U/ml penicillin-streptomycin. The cells were then plated onto collagen type I-coated flasks, and incubated at 37° C., room air with 5% CO₂. After 4 days, cell colonies began to sprout around the glomeruli. Cells showed an epithelial morphology with a polyhedral shape when confluency was reached at day 7. The cells were characterized as podocytes by detection of podocyte specific markers, synaptopodin and nephrin, by immunofluorescence staining.

siRNA Design and Transfection. Control siRNAs and siRNAs (antisense and sense strands) for thymosin β4 (Thym) were designed suggested by the manufacture (Invitrogen, San Diego, Calif., USA). The sense strand sequences for 4 different siRNAs and scrambled controls were as follows: Thym 1,5′-CCGATATGGCTGAGATCGAGAAATT; Thym 2,5′-GAG AAG CAA GCT GGC GAA TCG TAA T; Thym 3,5′-TCA AAG AAT CAG AAC TAC TGA GCA G; Thym 4 5′-GGG AGA TGA TGA AAT AGA GAG GAA A; control Thym 1,5′-CCG GGT AAG TCC TAG AGA GAT AAT T; control Thym 2,5′-GAT CCA TGC AGC GTA TCC GAT GAA T; control Thym 3,5′-TCA TAA GAG ACA TCA AGT CGA ACA G; control Thym 4, 5′-GGG ATG ATG AAA TAG AGA GGA GAA A. In vitro transfections were performed using Lipofecta™ 2000 Reagent (Invitrogen) according to the manufacturer's instructions. In brief, GEN were seeded on to 6-well plates one day prior to transfection. Transfection with siRNA was done when cells were above 50% confluent. 100 pmol of siRNA were used for 5×10⁵ cells in 2 ml of medium. Cells were washed 48 hrs after transfection. Angiotensin was used to stimulate GEN as a model of sclerosis mechanisms. GEN were stimulated with angiotensin II (Ang II, 10⁻⁶ M) for 24 hrs, with or without concomitant transfection with siRNA or control siRNA. Since all 4 designed siRNAs achieved equal downregulation of thymosin β4, only one set of siRNA and its scrambled control was used for these experiments. Results were compared to normal GEN as baseline control.

Western Blot Analysis. For thymosin β4 Western blot analysis, 100 μg of cell lysate from cultured GEN treated as above were separated by electrophoresis on 4-20% Tris-glycine gel (BioRad). Equal protein loading was confirmed by Coomassie blue staining of duplicate gels after electrophoresis. The gels were incubated for 1 hour in phosphate-buffered saline (PBS) containing 10% glutaraldehyde (Sigma), washed three times for 20 minutes in PBS, and further incubated in a blotting buffer containing 1× Tris-glycine transfer buffer (Invitrogen) and 20% methanol for 30 minutes at room temperature. Proteins were transferred to a nitrocellulose membrane (BioRad) by electrotransfer. The membrane was pre-incubated for 2 hours in PBS containing 5% skim milk and 0.05% Tween 20 (PBS-T), incubated for 1 hour at RT in PBS-T with specific antibody (rabbit polyclonal anti-thymosin β4, 1:1000; Biodesign), washed ×5 with PBS-T and incubated with horseradish peroxidase-conjugated secondary antibody (Amersham Biosciences, Buckinghamshire, UK) for 1 hour at RT. The membranes were washed ×5 with PBS-T, and bound antibody was detected with an enhanced chemiluminescence detection kit (Amersham Biosciences). Mouse spleen and muscle tissues were used as positive and negative controls, respectively.

For Western blot analysis of plasminogen activator inhibitor-1 (PAI-1), GEN cell lysate was separated by electrophoresis on 10% Tris-glycine gel (BioRad). After the transfer of protein, the membrane was incubated with antibody specific for sheep anti-mouse PAI-1 antibody (1:250, American Diagnostica Inc. Greenwich, Conn.). After incubation with horseradish peroxidase (HRP)-labeled anti-sheep IgG secondary antibody (1:1000 dilution in 5% milk TBS-T), the protein bands on Western blots were visualized as above and developed on film. The membranes were restripped for beta actin, used as a housekeeping control protein (Sigma) (Kerins et al., 1995).

Statistical Analysis. The statistical analyses of the proteomic data were done by the following three steps. First, the significant differentially expressed proteins from two different biological groups were selected. The protein was chosen as a significant protein marker if it met at least three of the six selection methods, which include Kruskal-Wallis test, Fisher's exact test, t-test, Significance Analysis of Microarrays (SAM), Weighted Gene Analysis (WGA) and the modified info score method. The cutoff points for each method were p<0.0001, p<0.0001, p<0.0001, 3.5, 2 and 0 respectively, which were determined based on the significance and the prediction power of each method. Second, the class prediction model was employed to assessed whether the patterns of protein expression could be used to classify tissue samples into two classes according to the selected protein markers based upon the Weighted Flexible Compound Covariate Method (WFCCM) (Shyr et al., 2003). Third, The misclassification rate was evaluated using the leave-one-out cross-validation class prediction method. The agglomerative hierarchical clustering algorithm was also applied to investigate the selected protein markers expression patterns as well as the classification accuracy for different biological samples using M. Eisen's software (Eisen et al., 1998).

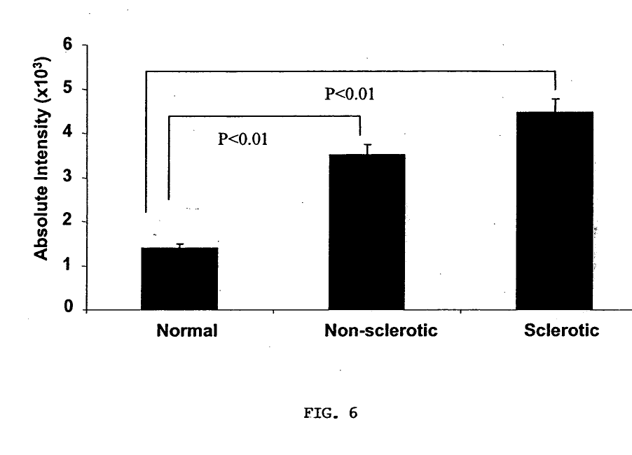

The proteomic pattern closeness comparison among the three groups was performed based on the following statistical method. Relative quantitative analysis of thymosin β4 was performed using the two-tailed student's t-test between the three classes of glomeruli. The average mass spectral intensity for thymosin β4 was obtained for each rat and the N number was 6 in the t-test analysis. The confidence level was 95%. The error bars in FIG. 6 were 95% confidence intervals. P values were obtained for the normal vs. nonscierotic glomeruli (p=0.015), normal vs. sclerotic glomeruli (p<0.05), and nonsclerotic vs. sclerotic glomeruli (p=0.4) comparisons.

### EXAMPLE 2

### Results

Protein Profiles Obtained Using MALDI-TOF Mass Spectrometry. Each MALDI mass spectrum was obtained from an average of five microdissected glomeruli averaging signals from 250 consecutive laser shots. From the six rats, a total of 60, 30, and 60 mass spectra were obtained from the normal, nonscierotic and sclerotic glomeruli, respectively. Approximately two hundred protein signals were detected per spectrum in the mass range of 2,000-70,000 Da, with the signals under 20,000 Da yielding the best resolution. Differentially expressed signals were found among the three different classes of glomeruli, as shown in FIG. 2.

Glomerular Proteomic Pattern Comparisons. 1473 distinct peaks across all the spectra were obtained. Using the weighted flexible compound covariate method (WFCCM) statistical analysis, we were able to classify the normal and sclerotic glomeruli proteomic pattern with 98.3% accuracy using the top 54 differentially expressed MS signals. Similarly, 96.7% classification accuracy was obtained for the comparison of normal glomeruli vs. nonsclerotic glomeruli using the top 166 differentially expressed MS signals. The inventors obtained 97.8% classification accuracy for nonsclerotic glomeruli from sclerotic glomeruli using the top 84 differentially expressed MS signals (Table 3).

TABLE 3
The weighted flexible compound covariant method (WFCCM) was
used to select the statistically significant peaks that allowed
classification of the three groups of samples according to their
proteomic patterns. The misclassification rate was calculated
using the leave-one-out cross-validation class prediction method.

| | No. of | |
|---|---|---|
| | Differentially | % Correct |
| Comparisons | Expressed Peaks | Classification |
| Normal Glomeruli | 54 | 98.3 |
| vs. Sclerotic | | |
| Glomeruli | | |
| Non-sclerotic | 166 | 96.7 |
| Glomeruli vs. | | |
| Normal Glomeruli | | |
| Non-sclerotic | 84 | 97.8 |
| Glomeruli vs. | | |
| Sclerotic Glomeruli | | |

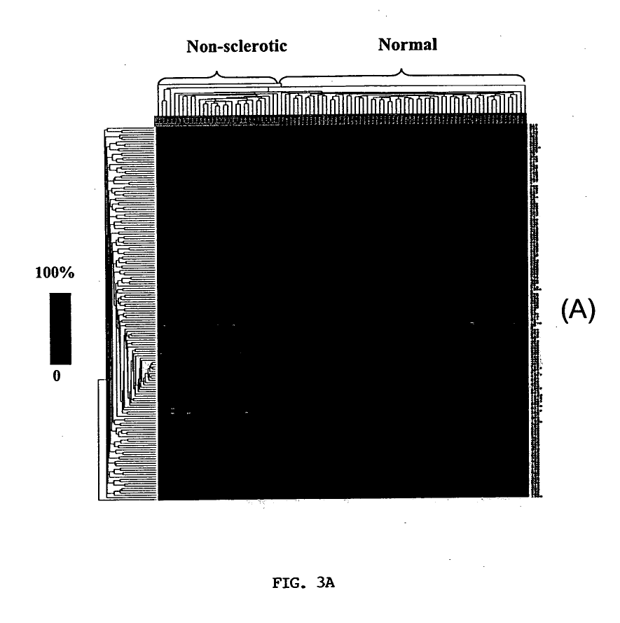

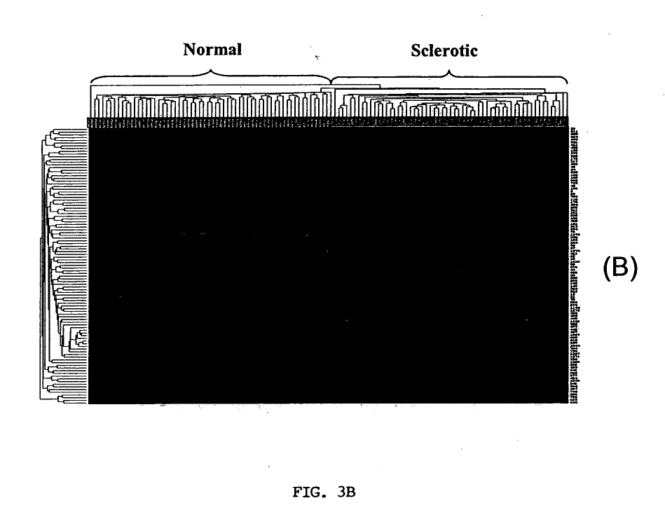

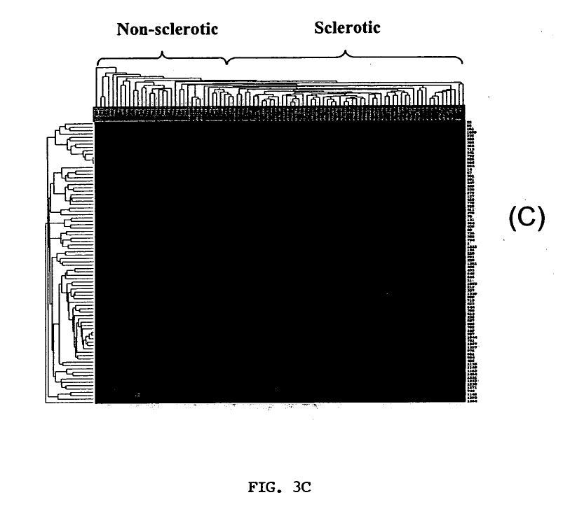

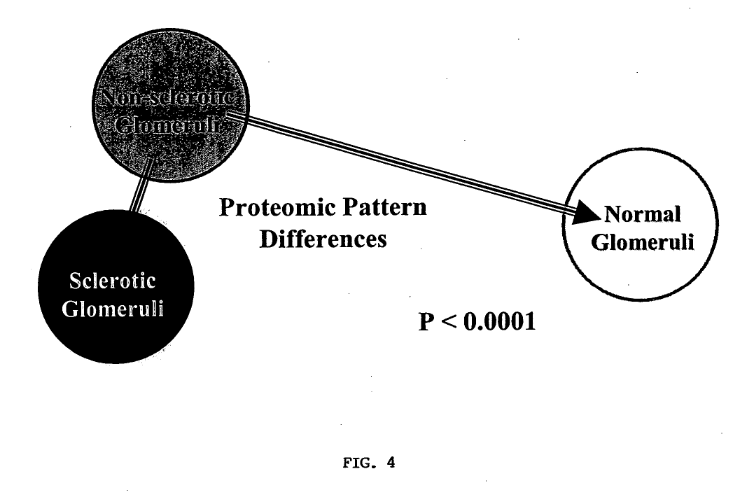

The agglomerative hierarchical clustering algorithm was used to investigate the protein expression patterns among the significant differentially expressed proteins with Eisen's software. The selected proteomic pattern distinguished all the normal glomeruli from nonsclerotic glomeruli with 100% classification accuracy. The inventors also obtained 99.2% classification accuracy in distinguishing normal vs. sclerotic glomeruli (1 protein profile out of 120 was misclassified) and 92.2% classification accuracy for nonsclerotic glomeruli vs. sclerotic glomeruli (7 protein profiles out of 90 were misclassified) (FIG. 3). The overall proteomic pattern of nonsclerotic glomeruli was more similar to sclerotic than to normal glomeruli (P<0.0001) (FIG. 4).

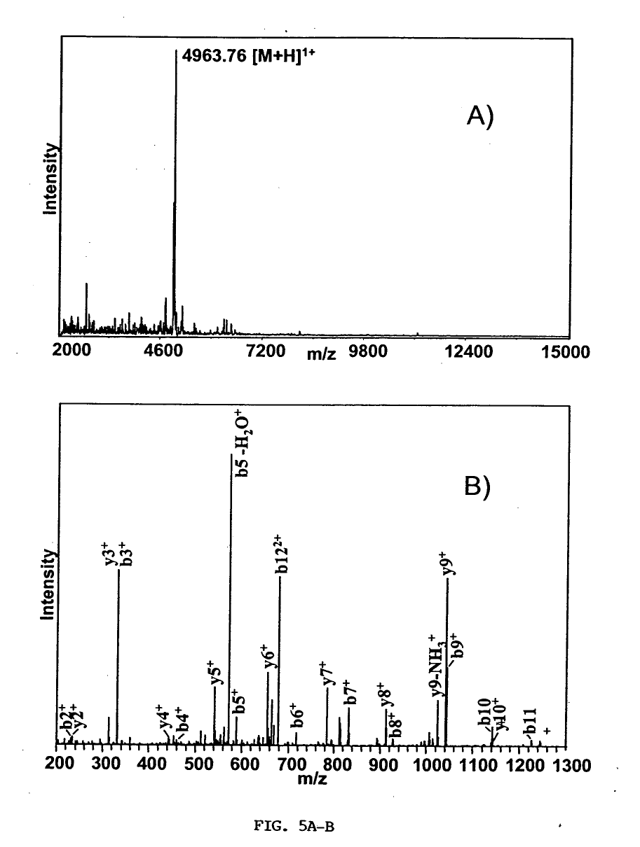

Identification of Thymosin β4. The protein markers that statistically most significantly contributed to differential classification of our three classes of glomeruli were targeted for identification. After the tissue homogenization and HPLC separation steps, three fractions containing the peak of m/z value 4963.76, one of the target proteins, were found using MALDI MS (FIG. 5A). With LC-MS/MS analysis of the resulting tryptic peptides, thymosin β4 was identified as the target protein marker. Multiple MS/MS spectra were found to be consistent with the thymosin β4 tryptic peptides: [TETQEKNPLPSK]₂₀₋₃₁, [KTETQEKNPLPSK]₁₉₋₃₁, [TETQEKNPLPSKETIEQEK]₂₀₋₃₈, and [KTETQEKNPLPSKETIEQEK]₁₉₋₃₈. The sequest cross correlation scores for these sequences were 3.42, 3.89, 4.95 and 4.69, respectively, showing a strong correlation between the MS/MS spectra and the amino acid sequences. As an example, the MS/MS spectra for [TETQEKNPLPSK]₂₀₋₃₁ is shown in FIG. 5B. These sequences composed 44% of the total amino acid sequence of thymosin β4. Considering the previous reported N-terminal acetylation (Stoekli et al., 2001), the theoretical average molecular weight for thymosin β4 is 4963.5 Da, which matches well with the target protein molecular weight. The signal intensities of thymosin β4 from the mass spectra for normal glomeruli, nonsclerotic glomeruli and sclerotic glomeruli are shown in FIG. 6. Thymosin β4 expression levels were increased approximately 3-fold from normal to sclerotic glomeruli based on the MS intensities.

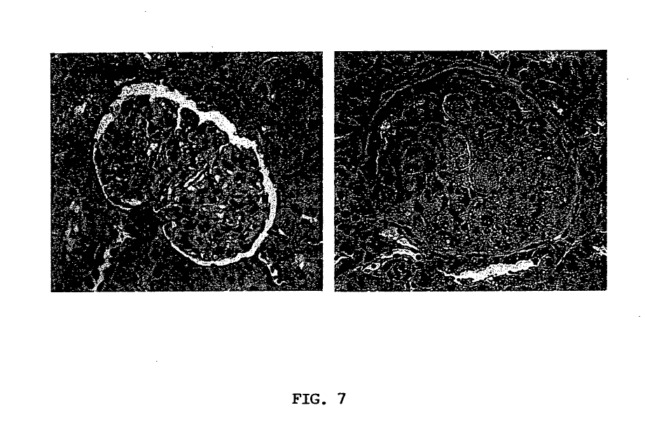

Thymosin B4 Expression In Vivo And In Vitro. Immunohistochemistry was performed to confirm the increased level of thymosin β4 in glomerulosclerosis. Thymosin β4 was increased in sclerotic glomeruli versus non-sclerotic or normal glomeruli (FIG. 7). Furthermore, thymosin β4 was found predominantly expressed in endothelial cells identified by serial section staining with RECA-1 (Abcam Inc, Cambridge, Mass.), while mesangial cells, stained with anti-rat Thy-1 (BD Pharmingen, San diego, Calif.) were negative for thymosin β4. Podocytes, identified anatomically, and macrophages, double-stained with EDI (Biosource) were also negative for thymosin β4.

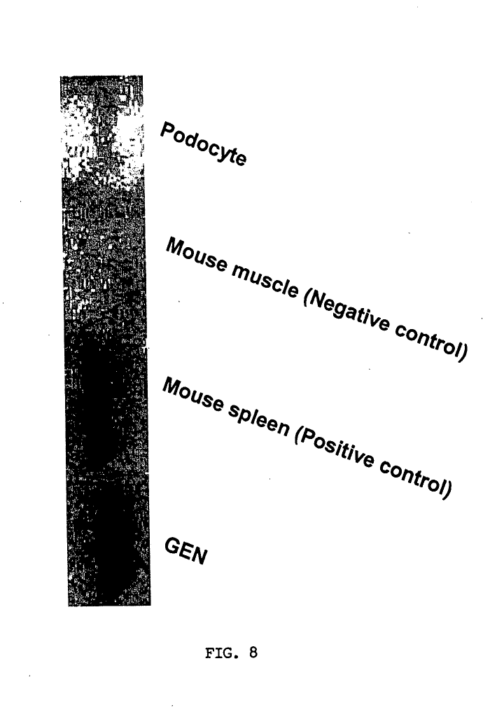

Further analyses were performed in vitro assessing thymosin β4 expression in two different glomerular cell lines (FIG. 8). Western blots from cultured glomerular endothelial (GEN) and podocyte cells confirmed endothelial expression of thymosin β4, with no protein detected in podocytes. GEN showed a strong immunoreactive band at 4.9 kDa corresponding to the expected molecular weight of thymosin β4. Mouse spleen and muscle, used as positive and negative controls of thymosin β4 immunohistochemistry and Western blot expression, showed expected results.

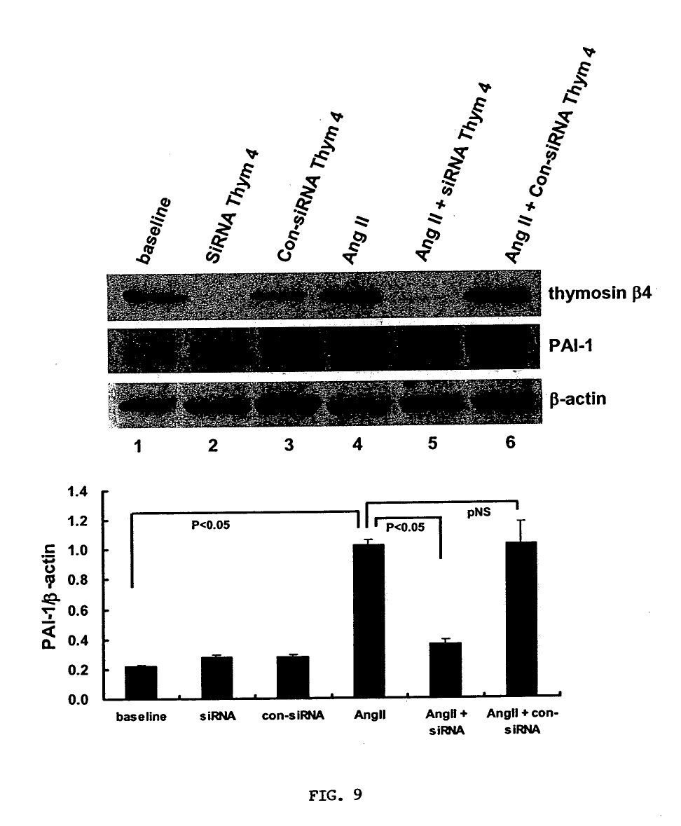

Thymosin β4 Effect on Sclerosis Mechanisms. The inventors next assessed whether modulation of thymosin β4 mRNA affected responses to Ang II. Angiotensin stimulates PAI-1 in vivo and in vitro (Kerins et al., 1995; Ma et al., 2000). The inventors therefore assessed PAI-1 expression in response to Ang II in GEN. To investigate the functional role of thymosin β4 in sclerosis, we designed siRNA and control siRNA for thymosin β4 and transfected them into GEN. Thymosin β4 protein expression was successfully knocked down by approximately 90% using siRNA, with equivalent efficacy of all four siRNAs tested. Scrambled control RNA had no effect. The inventors next assessed effect of downregulated thymosin β4 on the Ang II-induced prosclerotic response, by assessing expression of PAI-1 in these cells. Neither siRNA or control siRNA affected baseline PAI-1 expression. Angiotensin II (10⁻⁶ M) increased thymosin β4 over baseline, and concurrently dramatically upregulated PAI-1 in normal GEN, while transfection with siRNA for thymosin β4 significantly decreased the angiotensin II-induced PAI-1 expression. The control siRNA had no effect on the Ang II-induced PAI-1 expression (FIG. 9).

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

IX. References

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 4,683,195
U.S. Pat. No. 4,683,202
U.S. Pat. No. 4,684,611
U.S. Pat. No. 4,797,368
U.S. Pat. No. 4,800,159
U.S. Pat. No. 4,883,750
U.S. Pat. No. 4,952,500
U.S. Pat. No. 5,139,941
U.S. Pat. No. 5,242,974
U.S. Pat. No. 5,279,721
U.S. Pat. No. 5,302,523
U.S. Pat. No. 5,322,783
U.S. Pat. No. 5,384,253
U.S. Pat. No. 5,384,261
U.S. Pat. No. 5,405,783
U.S. Pat. No. 5,412,087
U.S. Pat. No. 5,424,186
U.S. Pat. No. 5,429,807
U.S. Pat. No. 5,436,327
U.S. Pat. No. 5,445,934
U.S. Pat. No. 5,464,765
U.S. Pat. No. 5,472,672
U.S. Pat. No. 5,527,681
U.S. Pat. No. 5,529,756
U.S. Pat. No. 5,532,128
U.S. Pat. No. 5,538,877
U.S. Pat. No. 5,538,880
U.S. Pat. No. 5,545,531
U.S. Pat. No. 5,550,318
U.S. Pat. No. 5,554,501
U.S. Pat. No. 5,556,752
U.S. Pat. No. 5,561,071
U.S. Pat. No. 5,563,055
U.S. Pat. No. 5,571,639
U.S. Pat. No. 5,580,859
U.S. Pat. No. 5,589,466
U.S. Pat. No. 5,591,616
U.S. Pat. No. 5,593,839
U.S. Pat. No. 5,599,695
U.S. Pat. No. 5,610,042
U.S. Pat. No. 5,624,711
U.S. Pat. No. 5,656,610
U.S. Pat. No. 5,658,734
U.S. Pat. No. 5,700,637
U.S. Pat. No. 5,702,932
U.S. Pat. No. 5,736,524
U.S. Pat. No. 5,780,448
U.S. Pat. No. 5,789,215
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,336
U.S. Pat. No. 5,882,864
U.S. Pat. No. 5,900,481
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,925,565
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,819
U.S. Pat. No. 5,935,825
U.S. Pat. No. 5,939,291
U.S. Pat. No. 5,942,391
U.S. Pat. No. 5,945,100
U.S. Pat. No. 5,981,274
U.S. Pat. No. 5,994,136
U.S. Pat. No. 5,994,624
U.S. Pat. No. 6,004,755
U.S. Pat. No. 6,013,516
Abbondanzo, Ann Diagn Pathol, 3(5):318-327, 1999.
Akis and Madaio, Kidney Int. 65:2223-2227, 2004.
Al-Nedawi et al., Blood 103:1319-1324, 2004.
Alizadeh et al., Nature 403 (6769), 503-511, 2000.
Allred et al., Arch Surg, 125(1):107-13, 1990.
Almendro et al., J Immunol., 157:5411-5421, 1996.
Angel et al., Mol. Cell. Biol., 7:2256, 1987.
Angel et al., Cell, 49:729, 1987b.
Angel et al., Mol. Cell. Biol., 7:2256, 1987a.
Antibodies: A Laboratory Manual, Cold Spring Harbor Laboratory, Cold Spring Harbor Press, Cold Spring Harbor, N.Y., 1988.
Arthur, J. M., Curr. Opin. Nephrol. Hypertens. 12:423-430, 2003.
Atchison and Perry, Cell, 46:253, 1986.
Atchison and Perry, Cell, 48:121, 1987.
Ausubel et al., In: Current Protocols in Molecular Biology, John, Wiley & Sons, Inc, New York, 1994.
Baichwal and Sugden, In: Gene Transfer, Kucherlapati (ed.), New York, Plenum Press, 117-148, 1986.
Banerji et al., Cell, 27(2 Pt 1):299-308, 1981.
Banerji et al., Cell, 33(3):729-740, 1983.
Ben-Dor et al., J. Comput. Biol. 7(3-4): 559-583, 2000.
Berkhout et al., Cell, 59:273-282, 1989.
Bhattacharya et al., J. Proteome Res. 2:95-98, 2003.
Bittner et al., Nature 406 (6795), 536-40, 2000.
Blanar et al., EMBO J., 8:1139, 1989.
Blomer et al., J. Virol., 71(9):6641-6649, 1997.
Bodine and Ley, EMBO J., 6:2997, 1987.
Borg and Groenen, In: Modern multidimensional scaling: Theory and applications. Springer, New York. 1997
Boshart et al., Cell, 41:521, 1985.
Bosze et al., EMBO J., 5(7):1615-1623, 1986.
Braddock et al., Cell, 58:269, 1989.
Brown et al., Immunol Ser, 53:69-82, 1990.
Bulla and Siddiqui, J. Virol., 62:1437, 1986.
Campbell and Villarreal, Mol. Cell. Biol., 8: 1993, 1988.
Campere and Tilghman, Genes and Dev., 3:537, 1989.
Campo et al., Nature, 303:77, 1983.
Carbonelli et al., FEMS Microbiol Lett, 177(1):75-82, 1999.
Celander and Haseltine, J. Virology, 61:269, 1987.
Celander et al., J Virology, 62:1314, 1988.
Chamberlan et al., In: PCR Protocols, eds. Innis, Gelfand, Sninsky, White (Academic Press, NY, 272-281, 1990.
Chandler et al., Cell, 33:489, 1983.
Chandler et al., Proc Natl Acad Sci USA, 94(8):3596-601, 1997.
Chang et al., Mol. Cell. Biol., 9:2153, 1989.
Chatterjee et al., Proc Natl. Acad. Sci. U.S.A., 86:9114, 1989.
Chaurand et al., Proteomics 3:2221-2239, 2003.
Chen and Okayama, Mol. Cell Biol., 7(8):2745-2752, 1987.
Choi et al., Cell, 53:519, 1988.
Cocea, Biotechniques, 23(5):814-816, 1997.
Cohen et al., J. Cell. Physiol., 5:75, 1987.
Costa et al., Mol Cell. Biol., 8:81, 1988.
Cotten et al., Proc Natl Acad Sci USA, 89(13):6094-6098, 1992.
Coupar et al., Gene, 68:1-10, 1988.
Cripe et al., EMBO J., 6:3745, 1987.
Culotta and Hamer, Mol. Cell. Biol., 9:1376, 1989.
Curiel, Nat Immun, 13(2-3):141-64, 1994.
Dandolo et al., J. Virology, 47:55-64, 1983.
Daumas-Duport et al., Cancer 62, 2152-2165, 1988.
De Jager et al., Semin Nucl Med 23(2):165-179, 1993.
De Villiers et al., Nature, 312(5991):242-246, 1984.
Deschamps et al., Science, 230:1174-1177, 1985.
Doolittle et al., Methods Mol. Biol., 109:215-237, 1999.
Dougherty, Comparative and Functional Genomics 2:28-34, 2001.
Edbrooke et al., Mol. Cell. Biol., 9:1908, 1989.
Eddy A. A., Am. J. Physiol. 283:F209-220, 2002.
Edlund et al., Science, 230:912-916, 1985.
Eisen et al., Proc. Nat'l Acad. Sci. USA 95:14863-14868, 1998.
Emmert-Buck et al., Science 274:998-1001, 1996.
Eng et al., J. Am. Soc. Mass. Spectrom. 5:976-989, 1994.
European Application No. 0 364 255
European Application No. 320 308
European Application No. EPO 0273085
European Application No. GB 2 202 328
European Application No. 329 822
Fechheimer et al., Proc. Nat'l Acad. Sci. USA, 84:8463-8467, 1987.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fogo et al., Kidney Int. 38:115-123, 1990.
Fogo, A. B. Kidney Int. 75:S15-21, 2000.
Fraley et al., Proc. Nat'l Acad. Sci. USA, 76:3348-3352, 1979.
Friedmann, Science, 244:1275-1281, 1989.
Frohman, In: PCR Protocols. A Guide To Methods And Applications, Academic Press, N.Y., 1990.
Fujita et al., Cell, 49:357, 1987.
Fuller et al., Cancer Res. 59, 4228-4232, 1999.
Ghosh and Bachhawat, In: Liver Diseases, Targeted Diagnosis and Therapy Using Specific Receptors and Ligands. Wu et al., eds., Marcel Dekker, New York, pp. 87-104, 1991.
Gilles et al., Cell, 33:717, 1983.
Gloss et al., EMBO J., 6:3735, 1987.
Godbout et al., Mol. Cell. Biol., 8:1169, 1988.
Golub et al., Science 286, 531-537, 1999.
Good et al., Am. J. Kidney Dis. 43:933-938, 2004.
Goodbourn and Maniatis, Proc. Nat'l Acad. Sci. USA, 85:1447, 1988.
Goodbourn et al., Cell, 45:601, 1986.
Gopal, Mol. Cell Biol., 5:1188-1190, 1985.
Graham and Van Der Eb, Virology, 52:456-467, 1973.
Grant et al., Angiogenesis 3:125-135, 1999.
Greene et al., Immunology Today, 10:272, 1989
Grosschedl and Baltimore, Cell, 41:885, 1985.
Grunhaus et al., Seminar in Virology, 200(2):535-546, 1992.
Gulbis and Galand, Hum Pathol 24(12):1271-1285, 1993.
Harland and Weintraub, J. Cell Biol., 101: 1094-1099, 1985.
Haslinger and Karin, Proc. Nat'l Acad. Sci. USA, 82:8572, 1985.
Hauber and Cullen, J. Virology, 62:673, 1988.
Hedenfalk et al., N Engl J Med 244, 539-548, 2001.
Hen et al., Nature, 321:249, 1986.
Hensel et al., Lymphokine Res., 8:347, 1989.
Herr and Clarke, Cell, 45:461, 1986.
Hirochika et al., J. Virol., 61:2599, 1987.
Hirsch et al., Mol. Cell. Biol., 10:1959, 1990.
Holbrook et al., Virology, 157:211, 1987.
Horlick and Benfield, Mol. Cell. Biol., 9:2396, 1989.
Horwich et al., Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Ikoma et al. Kidney Int. 40:195-202, 1991.
Imagawa et al., Cell, 51:251, 1987.
Imbra and Karin, Nature, 323:555, 1986.
Imler et al., Mol. Cell. Biol, 7:2558, 1987.
Imperiale and Nevins, Mol. Cell. Biol., 4:875, 1984.
Inouye and Inouye, Nucleic Acids Res., 13: 3101-3109, 1985.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Kaeppler et al., Plant Cell Reports 9: 415-418, 1990.
Kaneda et al., Science, 243:375-378, 1989.
Karin et al., Mol. Cell. Biol., 7:606, 1987.
Katinka et al., Cell, 20:393, 1980.
Kato et al., J. Biol. Chem., 266:3361-3364, 1991.
Kawamoto et al., Mol. Cell. Biol., 8:267, 1988.
Kelleher and Vos, Biotechniques, 17(6):1110-7, 1994.
Kendall and Gibbons, Rank Correlation Methods, Fifth Edition. Oxford University Press, New York. 1990.
Kerins et al., J. Clin. Invest. 96:2515-2520, 1995.
Khan et al., Nature Medicine 7(6): 673-679, 2001.
Kiledjian et al., Mol. Cell. Biol., 8:145, 1988.
Kim et al., J. Comput. Biol. 9:129-148, 2002.
Klamut et al., Mol. Cell. Biol., 10:193, 1990.
Kleihues and Cavenee, World Health Organzation Classification of Tumours of the Nervous System,. New York: Oxford University Press, 2000.
Klein and Thongboonkerd, Contrib. Nephrol. 141:1-10, 2004.
Klein et al., Nature, 327:70-73, 1987.
Koch et al., Mol. Cell. Biol., 9:303, 1989.
Kraus et al. FEBS Lett., 428(3): 165-170, 1998.
Kriegler and Botchan, In: Eukaryotic Viral Vectors, Y. Gluzman, ed., Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Kwoh et al., Proc. Nat. Acad. Sci. USA, 86: 1173, 1989.
Lareyre et al., J Biol Chem., 274(12):8282-8290, 1999.
Larsen et al., Proc Natl. Acad. Sci. USA., 83:8283, 1986.
Laspia et al., Cell, 59:283, 1989.
Latimer et al., Mol. Cell. Biol., 10:760, 1990.
Laughlin et al., J. Virol., 60(2):515-524, 1986.
Lebkowski et al., Mol. Cell. Biol., 8(10):3988-3996, 1988.
Lee et al., J Auton Nerv Syst. 74(2-3):86-90, 1997.
Lee et al., Nature, 294:228, 1981.
Lee et al., Nucleic Acids Res., 12:4191-206, 1984.
Levenson et al., Hum Gene Ther, 9(8):1233-6, 1998.
Levinson et al., Nature, 295:79, 1982.
Lin et al., Mol. Cell. Biol., 10:850, 1990.
Low & Goldstein, Biol. Chem. 257(2):1000-6, 1982.
Luria et al., EMBO J., 6:3307, 1987.
Lusky and Botchan, Proc Natl. Acad. Sci. U.S.A., 83:3609, 1986.
Lusky et al., Mol. Cell. Biol. 3:1108, 1983.
Ma et al., Kidney Int. 58:2425-2436, 2000.
Ma & Fogo, Semin. Nephrol. 21:544-553, 2001.
Macejak and Sarnow, Nature, 353:90-94, 1991.
Majors and Varmus, Proc. Nat'l Acad. Sci. USA, 80:5866, 1983.
Malinda et al., J. Invest. Dermatol. 113(3):364-8, 1999.
Maniatis, et al., Molecular Cloning, A Laboratory Manual, Cold Spring Harbor Press, Cold Spring Harbor, N.Y., 1989.
Mann et al., Cell, 33:153-159, 1983.
McLaughlin et al., J. Virol., 62(6):1963-1973, 1988.
McNeall et al., Gene, 76:81, 1989.
Miksicek et al., Cell, 46:203, 1986.
Miller et al., Am. J. Clin. Oncol., 15(3):216-221, 1992.
Mora et al., Int. J. Immunopharmacol. 19:1-8, 1997.
Mordacq and Linzer, Genes and Dev., 3:760, 1989.
Moreau et al., Nuc. Acids Res., 9:6047, 1981.
Mueller and B. Wold, Science 246, 780-786, 1989.
Muesing et al., Cell, 48:691, 1987.
Muzyczka, Curr Top Microbiol Immunol, 158:97-129, 1992.
Nabel and Baltimore, Nature 326:711-713, 1987.
Nakamura et al., In: Handbook of Experimental Immunology (4^{th} Ed.), Weir et al., (eds). 1:27, Blackwell Scientific Publ., Oxford, 1987.
Naldini et al., Science, 272(5259):263-267, 1996.
Ng et al., Nuc. Acids Res., 17:601, 1989.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al, Methods Enzymol., 149:157-176, 1987.
Nomoto et al., Gene, 236(2):259-271, 1999.
Ohara et al., Proc. Nat'l Acad. Sci. USA, 86: 5673-5677, 1989.
Omirulleh et al., Plant Mol. Biol., 21:415-28, 1993.
Ondek et al., EMBO J., 6:1017, 1987.
Ornitz et al., Mol. Cell. Biol., 7:3466, 1987.
Palmer-Toy et al., Clin. Chem. 46:1513-1516, 2000.
Palmiter et al, Nature, 300:611, 1982.
Paskind et al., Virology, 67:242-248, 1975.
PCT Application PCT/US87/00880
PCT Application PCT/US89/01025
PCT Application WO 88/10315
PCT Application WO 90/07641
PCT Application WO89/06700
Pech et al., Mol. Cell. Biol., 9:396, 1989.
Pelletier and Sonenberg, Nature, 334:320-325, 1988.
Perales et al., Proc. Nat'l Acad. Sci. USA, 91:4086-4090, 1994.
Perez-Stable and Constantini, Mol. Cell. Biol., 10:1116, 1990.
Perou et al., Nature 406 (6797), 747-752, 2000.
Picard and Schaffner, Nature, 307:83, 1984.
Pinkert et al., Genes and Dev., 1:268, 1987.
Ponta et al., Proc. Nat'l Acad. Sci. USA, 82:1020, 1985.
Porton et al., Mol. Cell. Biol., 10: 1076, 1990.
Potrykus et al., Mol. Gen. Genet., 199:183-188, 1985.
Potter et al., Proc. Nat'l Acad. Sci. USA, 81:7161-7165, 1984.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Resendez Jr. et al., Mol. Cell. Biol., 8:4579, 1988.
Ridgeway, In: Vectors: A survey of molecular cloning vectors and their uses, Stoneham: Butterworth, pp. 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Roux et al., Proc. Nat'l Acad. Sci. USA, 86:9079-9083, 1989.
Safer et al., Proc. Nat'l Acad. Sci. USA 87:2536-2540, 1990.
Sakai et al., Genes and Dev., 2:1144, 1988.
Sambrook et al., In: Molecular Cloning: A Laboratory Manual, 2nd Ed., Cold Spring Harbor Press, Cold Spring Harbor, N.Y., 1989.
Sanders et al., Proc. Nat'l Acad. Sci. USA 89(10):4678-82 1992.
Satake et al., J. Virology, 62:970, 1988.
Schaffner et al., J. Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J., 6:1913, 1987.
Sherman et al., Mol. Cell. Biol., 9:50, 1989.
Shyr and Kim, Practical Approach to Microarray Data Analysis 186-200, 2003.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stephens and Hentschel, Biochem. J., 248:1, 1987.
Sterling et al., How to Build a Beowulf: A Guide to the Implementation and Application of PC Clusters, The MIT Press, Cambridge, Mass. 1999.
Stoeckli et al., Nat. Med. 7:493-496, 2001.
Stuart et al., Nature, 317:828, 1985.
Sullivan and Peterlin, Mol. Cell. Biol., 7:3315, 1987.
Swartzendruber and Lehman, J. Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10: 165, 1990.
Taylor and Kingston, Mol. Cell. Biol., 10: 176, 1990.
Taylor et al., J. Biol. Chem., 264:15160, 1989.
Temin, In: Gene Transfer, Kucherlapati (ed.), New York: Plenum Press, pp. 149-188, 1986.
Thiesen et al., J. Virology, 62:614, 1988.
Tratschin et al., Mol. Cell. Biol., 4:2072-2081, 1984.
Treisman, Cell, 42:889, 1985.
Tronche et al., Mol. Biol. Med., 7:173, 1990.
Trudel and Constantini, Genes and Dev. 6:954, 1987.
Tsumaki et al., J Biol Chem. 273(36):22861-22864, 1998.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Vasseur et al., Proc Natl. Acad. Sci. U.S.A., 77:1068, 1980.
Wagner et al., Science, 260:1510-1513, 1993.
Walker et al., Proc. Nat'l Acad. Sci. USA, 89:392-396 1992.
Wang and Calame, Cell, 47:241, 1986.
Weber et al., Cell, 36:983, 1984.
Weinberger et al. Mol. Cell. Biol., 8:988, 1984.
Wilson et al., Science, 244:1344-1346, 1989.
Winoto and Baltimore, Cell 59:649, 1989.
Wong et al., Gene, 10:87-94, 1980.
Wu and Wu, J. Biol. Chem., 262:4429-4432, 1987.
Wu and Wu, Adv. Drug Delivery Rev., 12:159-167, 1993.
Wu and Wu, Biochemistry, 27:887-892, 1988.
Wu et al., Biochem. Biophys. Res. Commun., 233(1):221-226, 1997.
Xu et al., J. Am. Soc. Mass. Spectrom. 13:1292-1297, 2002.
Yang et al., Proc. Nat'l Acad. Sci. USA, 87:9568-9572, 1990.
Young et al. Nat. Med. 5(12):1424-7, 1999.
Yutzey et al. Mol. Cell. Biol., 9:1397, 1989.
Zhao-Emonet et al., Biochem. Biophys. Acta., 1442(2-3):109-19, 1998.
Zufferey et al., Nat Biotechnol, 15(9):871-875, 1997.

What is claimed is:

1. A method of classifying glomerular tissue as normal, non-sclerotic or sclerotic comprising:
(a) obtaining a glomerular tissue sample;
(b) analyzing protein content of said tissue sample;
(c) comparing the protein content of said tissue sample with a predetermined standard; and
(d) classifying said glomerular tissue as normal, non-sclerotic or sclerotic.

2. The method of claim 1, wherein said glomerular tissue is classified as focal segmental glomerulosclerotic.

3. The method of claim 1, wherein analyzing protein content comprises assessing proteomic patterns.

4. The method of claim 3, wherein analyzing comprises mass spectrometry.

5. The method of claim 3, wherein analyzing comprises immunohistochemistry.

6. The method of claim 3, wherein analyzing comprises 2-D gel electrophoresis.

7. The method of claim 1, wherein analyzing protein content comprises assessing thymosin β4 expression.

8. The method of claim 7, wherein analyzing comprises mass spectrometry.

9. The method of claim 7, wherein analyzing comprises immunohistochemistry.

10. The method of claim 1, wherein analyzing protein content comprises laser capture microdissection coupled with matrix-assisted laser desporption/ionization time-of-flight mass spectrometry.

11. The method of claim 1, wherein analyzing comprises assessing expression of one or more proteins having molecular weights of 4222 Daltons, 5485 Daltons, 7018 Daltons and 12,131 Daltons.

12. The method of claim 11, wherein analyzing comprises mass spectrometry.

13. The method of claim 1, wherein said sample is from a mammal.

14. The method of claim 1, wherein said sample is from a human.

15. The method of claim 1, further comprising making a medical treatment decision based on said classification.

16. The method of claim 7, further comprising analyzing assessing expression of one or more proteins having molecular weights of 4222 Daltons, 5485 Daltons, 7018 Daltons and 12,131 Daltons.

17. A method of identifying a marker in a glomerular tissue comprising:
(a) obtaining a diseased glomerular tissue sample;
(b) analyzing protein content of said tissue sample;
(c) comparing the protein content of said tissue sample with a normal glomerular tissue sample; and
(d) identifying a marker in said diseased glomerular tissue sampel that is as normal, non-sclerotic or sclerotic.

18. The method of claim 17, wherein analyzing comprises mass spectrometry, immunohistochemistry or 2-D gel electrophoresis.
